(19) United States
(12) Patent Application Publication
(10) Pub. No.: US 2006/0252964 A1
(43) Pub. Date: Nov. 9, 2006

(54) Process for the preparation of aromatic amines

(76) Inventors: Andrew John Blacker, West Yorkshire (GB); Juliette Martin, Tourlaville (FR)

Correspondence Address: MORGAN LEWIS & BOCKIUS LLP, 1111 PENNSYLVANIA AVENUE NW, WASHINGTON, DC, 20004, US

(21) Appl. No.: 10/560,305

(22) PCT Filed: Jun. 9, 2004
(86) PCT No.: PCT/GB04/02478

§ 371 Date: May 5, 2006

(30) Foreign Application Priority Data
Jun. 13, 2003 (GB) 0313661.1

(51) Int. Cl. C07C 209/14

(52) U.S. Cl. 564/402; 558/46

(57) ABSTRACT

There is provided a process for the preparation of aromatic amines of Formula (1): 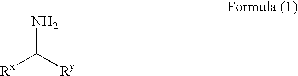
where R^{x} is optionally substituted aryl and R^{y} is optionally substituted hydrocarbyl, which comprises:
(a) reducing a compound of Formula (2): 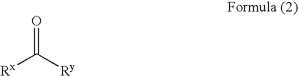
to give a compound of Formula (3): 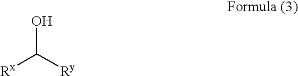
then, (b) reacting a compound of Formula (3) with a leaving group donor, to give a compound of Formula (4); 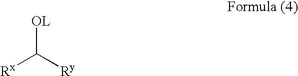 and, (c) reacting a compound of Formula (4) with ammonia to give the compound of Formula (1).

This invention relates to processes for the preparation of chiral aromatic amines and to novel substituted chiral aromatic amines.

Enantiomers of aromatic amines, such as 1-naphthylethylamine are valuable building blocks in the preparation of pharmaceutical and agrochemical active agents. They are also used as resolving agents for crystallisation/resolution of acidic species and as a chiral auxiliary.

According to a first aspect of the present invention there is provided a process for the preparation of a compound of Formula (1): 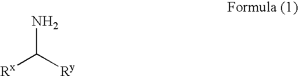
wherein:

R^{x} is optionally substituted aryl; and

R^{y} is optionally substituted hydrocarbyl:

which comprises the steps:

(a) reducing a compound of Formula (2): 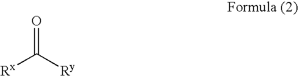
   to a compound of Formula (3): 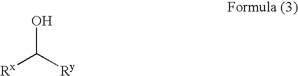
   wherein R^{x} and R^{y} are as defined for Formula (1):
(b) reacting a compound of Formula (3) with a leaving group donor, to give a compound of Formula (4); 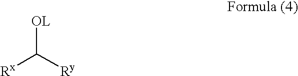
   wherein:

R^{x} and R^{y} are as defined for Formula (1); and

OL is a leaving group:
(c) reacting a compound of Formula (4) with ammonia to give a compound of Formula (1).

Preferably R^{y} is optionally substituted alkyl, optionally substituted alkenyl, optionally substituted alkynyl, optionally substituted aryl, optionally substituted heterocyclyl or any combination thereof.

When R^{y} comprises optionally substituted alkyl, optionally substituted alkenyl, or optionally substituted alkynyl it may be a linear, branched or cyclic molecule.

It is particularly preferred that R^{y} is optionally substituted alkyl, especially optionally substituted C₁₋₄alkyl, particularly C₁₋₄alkyl and more particularly methyl.

R^{x} is preferably optionally substituted phenyl or optionally substituted napthyl more preferably R^{x} is optionally substituted napthyl.

In many embodiments R^{x} and R^{y} are different.

Thus, there is preferably provided a process for the preparation of a compound of Formula (5): 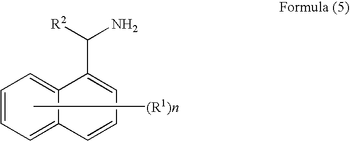
wherein:

R¹ is a substituent;

R² is optionally substituted hydrocarbyl; and

n is 0 to 4:

which comprises the steps:

(a) reducing a compound of Formula (6): 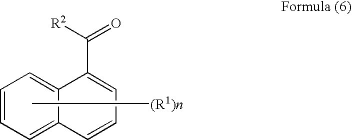
   to a compound of Formula (7): 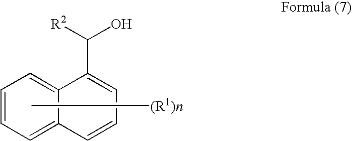
   wherein R¹, R² and n are as defined for Formula (5):
(b) reacting a compound of Formula (7) with a leaving group donor, to give a compound of Formula (8); 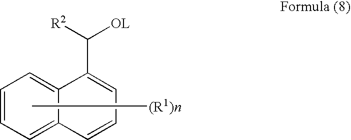
   wherein:

R¹, R² and n are as defined for Formula (5);

OL is a leaving group:
(c) reacting a compound of Formula (8) with ammonia to give a compound of Formula (5).

Preferably R² is optionally substituted alkyl, optionally substituted alkenyl, optionally substituted alkynyl, optionally substituted aryl, optionally substituted heterocyclyl or any combination thereof.

When R² comprises optionally substituted alkyl, optionally substituted alkenyl, or optionally substituted alkynyl it may be a linear, branched or cyclic molecule.

It is particularly preferred that R² is optionally substituted alkyl, especially optionally substituted C₁₋₄alkyl, particularly C₁₋₄-alkyl and more particularly methyl.

In the compounds of Formulae (1) to (8) the optional substituents on R^{y} and R² are preferably independently selected from: optionally substituted alkoxy (preferably C₁₋₄-alkoxy), optionally substituted aryl (preferably phenyl), optionally substituted aryloxy (preferably phenoxy), optionally substituted heterocyclyl, polyalkylene oxide (preferably polyethylene oxide or polypropylene oxide), carboxy, phosphato, sulfo, nitro, cyano, halo, ureido, —SO₂F, hydroxy, ester, —NR^{a}R^{b}, —COR^{a}, —CONR^{a}R^{b}, —NHCOR^{a}, carboxyester, sulfone, and —SO₂NR^{a}R^{b} wherein R^{a} and R^{b} are each independently H or optionally substituted alkyl (especially C₁₋₄-alkyl). Optional substituents for any of the substituents described for R^{y} and R² may be selected from the same list of substituents.

In the compounds of Formulae (1) to (8) the optional substituents on R^{x} and substituents R¹ are preferably independently selected from: optionally substituted alkyl (preferably C₁₋₄-alkyl), optionally substituted alkenyl (preferably C₁₋₄-alkenyl), optionally substituted alkynyl (preferably C₁₋₄-alkynyl), optionally substituted alkoxy (preferably C₁₋₄-alkoxy), optionally substituted aryl (preferably phenyl), optionally substituted aryloxy (preferably phenoxy), optionally substituted heterocyclyl, polyalkylene oxide (preferably polyethylene oxide or polypropylene oxide), carboxy, phosphato, sulfo, nitro, cyano, halo, ureido, —SO₂F, hydroxy, ester, —NR^{a}R^{b}, —COR^{a}, —CONR^{a}R^{b}, —NHCOR^{a}, carboxyester, sulfone, and —SO₂NR^{a}R^{b} wherein R^{a} and R^{b} are each independently H or optionally substituted alkyl (especially C₁₋₄-alkyl). Optional substituents for any of the above substituents may be selected from the list of substituents preferred for R^{y} and R².

Preferably n is 0.

The reduction of the keto group in a compound of Formula (2) or Formula (5) in step (a) may be carried out using any suitable method known in the art. These methods are summarised in Larock R. C., Comprehensive Organic Transformations, VCH, pages 527 to 548 which is included herein by reference and include reduction with: LiAlH₄, diisobutyl aluminium hydride (DIBAL), NaBH₄ or BH₃; reduction by a biological system, such as an enzyme or a microbial cell or cell preparation; or reduction using a Nobel metal or Raney catalyst such as Pt in the presence of hydrogen.

Step (a) is preferably carried out in the presence of a catalyst.

Catalysts include transfer hydrogenation catalysts such as: (a) the chiral Ruthenium (II) catalysts developed for ketone reduction which are disclosed in Chem. Rev., 1998, 98, 2607 see Table 2; (b) the Zhang tridentate bis(oxazolinylmethyl)amine catalysts and related catalysts as disclosed in J. Am. Chem. Soc., 1998, 120, 3817, Tet. Let., 1997, 38(37), 6565 and in WO99/24410 (particularly the bis(phenyloxazolin-2-yl)amine and related catalysts discussed therein); and (c) the transition metal, particularly group VIII metal, complexes with chiral ligands of formula:

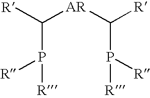

wherein AR is any aromatic or ring structure and R′, R″ and R′″ are each independently selected from aryl, alkyl, aralkyl, ring-substituted aralkyl, substituted aryl and combinations thereof as disclosed in U.S. Pat. No. 5,767,276, the catalysts of (a), (b) and (c) being incorporated herein by reference.

However, in a preferred embodiment step (a) is a transfer hydrogenation carried out using a hydrogen donor and a catalyst as described in International Patent Applications WO 98/42643, WO 00/18708 and WO 01/12574 which references are incorporated herein, in their entirety, by reference.

The preferred transfer hydrogenation catalysts for use In the process of the present invention are of general Formula (A): 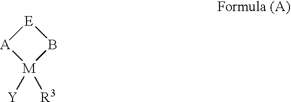
wherein:

R³ represents a neutral optionally substituted hydrocarbyl, a neutral optionally substituted perhalogenated hydrocarbyl, or an optionally substituted cyclopentadienyl ligand;

A represents —NR⁴—, —NR⁵—, —NHR⁴, —NR⁴R⁵ or —NR⁵R⁶ where R⁴ is H, C(O)R⁶, SO₂R⁶, C(O)NR⁶R¹⁰, C(S)NR⁶R¹⁰, C(═NR¹⁰)SR¹¹ or C(═NR¹⁰)OR¹¹, R⁵ and R⁶ each independently represents an optionally substituted hydrocarbyl, perhalogenated hydrocarbyl or an optionally substituted heterocyclyl group, and R¹⁰ and R¹¹ are each independently hydrogen or a group as defined for R⁶;

B represents —O—, —OH, OR⁷, —S—, —SH, SR⁷, —NR⁷—, —NR⁸—, —NHR⁸, —NR⁷R⁸, —NR⁷R⁹, —PR⁷— or —PR⁷R⁹ where R⁸ is H, C(O)R⁹, SO₂R⁹, C(O)NR⁹R¹², C(S)NR⁹R¹², C(═NR¹²)SR¹³ or C(═NR¹²)OR¹³, R⁷ and R⁹ each independently represents an optionally substituted hydrocarbyl, perhalogenated hydrocarbyl or an optionally substituted heterocyclyl group, and R¹² and R¹³ are each independently hydrogen or a group as defined for R⁹;

E represents a linking group;

M represents a metal capable of catalysing transfer hydrogenation; and

Y represents an anionic group, a basic ligand or a vacant site;

provided that when Y is not a vacant site that at least one of A or B carries a hydrogen atom.

The catalytic species is believed to be substantially as represented in the above formula. It may be introduced on a solid support.

Optionally substituted hydrocarbyl groups represented by R⁵⁻⁷ or R⁹⁻¹¹ include alkyl, alkenyl, alkynyl and aryl groups, and any combination thereof, such as aralkyl and alkaryl, for example benzyl groups.

Alkyl groups which may be represented by R⁵⁻⁷ or R⁹⁻¹¹ include linear and branched alkyl groups comprising 1 to 20 carbon atoms, particularly from 1 to 7 carbon atoms and preferably from 1 to 5 carbon atoms. In certain embodiments, the alkyl group may be cyclic, commonly comprising from 3 to 10 carbon atoms in the largest ring and optionally featuring one or more bridging rings. Examples of alkyl groups which may be represented by R⁵⁻⁷ or R⁹⁻¹¹ include methyl, ethyl, propyl, 2-propyl, butyl, 2-butyl, t-butyl and cyclohexyl groups.

Alkenyl groups which may be represented by one or more of R⁵⁻⁷ or R⁹⁻¹¹ include C₂₋₂₀, and preferably C₂₋₆ alkenyl groups. One or more carbon-carbon double bonds may be present. The alkenyl group may carry one or more substituents, particularly phenyl substituents.

Alkynyl groups which may be represented by one or more of R⁵⁻⁷ or R⁹⁻¹¹ include C₂₋₂₀, and preferably C₂₋₁₀ alkynyl groups. One or more carbon-carbon triple bonds may be present. The alkynyl group may carry one or more substituents, particularly phenyl substituents. Examples of alkynyl groups include ethynyl, propyl and phenylethynyl groups.

Aryl groups which may be represented by one or more of R⁵⁻⁷ or R⁹⁻¹¹ may contain 1 ring or 2 or more fused or bridged rings which may include cycloalkyl, aryl or heterocyclic rings. Examples of aryl groups which may be represented by R⁵⁻⁷ or R⁹⁻¹¹ include phenyl, tolyl, fluorophenyl, chlorophenyl, bromophenyl, trifluoromethylphenyl, anisyl, naphthyl and ferrocenyl groups.

Perhalogenated hydrocarbyl groups which may be represented by one or more of R⁵⁻⁷ or R⁹⁻¹¹ independently include perhalogenated alkyl and aryl groups, and any combination thereof, such as aralkyl and alkaryl groups. Examples of perhalogenated alkyl groups which may be represented by R⁵⁻⁷ or R⁹⁻¹¹ include —CF₃ and —C₂F₅.

Heterocyclic groups which may be represented by one or more of R⁵⁻⁷ or R⁹⁻¹¹ independently include aromatic, saturated and partially unsaturated ring systems and may comprise 1 ring or 2 or more fused rings which may include cycloalkyl, aryl or heterocyclic rings. The heterocyclic group will contain at least one heterocyclic ring, the largest of which will commonly comprise from 3 to 7 ring atoms in which at least one atom is carbon and at least one atom is any of N, O, S or P. Examples of heterocyclic groups which may be represented by R⁵⁻⁷ or R⁹⁻¹¹ include pyridyl, pyrimidyl, pyrrolyl, thiophenyl, furanyl, indolyl, quinolyl, isoquinolyl, imidazolyl and triazolyl groups.

When any of R⁵⁻⁷ or R⁹⁻¹¹ is a substituted hydrocarbyl or heterocyclic group, the substituent(s) should be such so as not to adversely affect the rate or stereoselectivity of the reaction. Optional substituents include halogen, cyano, nitro, hydroxy, amino, imino, thiol, acyl, hydrocarbyl, perhalogenated hydrocarbyl, heterocyclyl, hydrocarbyloxy, mono or di-hydrocarbylamino, hydrocarbylthio, esters, carboxy, carbonates, amides, sulphonyl and sulphonamido groups wherein the hydrocarbyl groups are as defined for R⁵⁻⁷ or R⁹⁻¹¹ above. One or more substituents may be present. R⁵⁻⁷ or R⁹⁻¹¹ may each contain one or more chiral centres.

The neutral optionally substituted hydrocarbyl or perhalogenated hydrocarbyl ligand which may be represented by R³ includes optionally substituted aryl and alkenyl ligands.

Optionally substituted aryl ligands which may be represented by R³ may contain 1 ring or 2 or more fused rings which include cycloalkyl, aryl or heterocyclic rings. Preferably, the ligand comprises a 6 membered aromatic ring. The ring or rings of the aryl ligand are often substituted with hydrocarbyl groups. The substitution pattern and the number of substituents will vary and may be influenced by the number of rings present, but often from 1 to 6 hydrocarbyl substituent groups are present, preferably 2, 3 or 6 hydrocarbyl groups and more preferably 6 hydrocarbyl groups. Preferred hydrocarbyl substituents include methyl, ethyl, iso-propyl, menthyl, neomenthyl and phenyl. Particularly when the aryl ligand is a single ring, the ligand is preferably benzene or a substituted benzene. When the ligand is a perhalogenated hydrocarbyl, preferably it is a polyhalogenated benzene such as hexachlorobenzene or hexafluorobenzne. When the hydrocarbyl substitutents contain enantiomeric and/or diastereomeric centres, it is preferred that the enantiomerically and/or diastereomerically purified forms of these are used. Benzene, p-cymyl, mesitylene and hexamethylbenzene are especially preferred ligands.

Optionally substituted alkenyl ligands which may be represented by R³ include C₂₋₃₀, and preferably C₆₋₁₂, alkenes or cycloalkenes with preferably two or more carbon-carbon double bonds, preferably only two carbon-carbon double bonds. The carbon-carbon double bonds may optionally be conjugated to other unsaturated systems which may be present, but are preferably conjugated to each other. The alkenes or cycloalkenes may be substituted preferably with hydrocarbyl substituents. When the alkene has only one double bond, the optionally substituted alkenyl ligand may comprise two separate alkenes. Preferred hydrocarbyl substituents include methyl, ethyl, iso-propyl and phenyl. Examples of optionally substituted alkenyl ligands include cyclo-octa-1,5-diene and 2,5-norbornadiene. Cyclo-octa-1,5-diene is especially preferred.

Optionally substituted cyclopentadienyl groups which may be represented by R³ include cyclopentadienyl groups capable of eta-5 bonding. The cyclopentadienyl group is often substituted with from 1 to 5 hydrocarbyl groups, preferably with 3 to 5 hydrocarbyl groups and more preferably with 5 hydrocarbyl groups. Preferred hydrocarbyl substituents include methyl, ethyl and phenyl. When the hydrocarbyl substitutents contain enantiomeric and/or diastereomeric centres, it is preferred that the enantiomerically and/or diastereomerically purified forms of these are used. Examples of optionally substituted cyclopentadienyl groups include cyclopentadienyl, pentamethyl-cyclopentadienyl, pentaphenylcyclopentadienyl, tetraphenylcyclopentadienyl, ethyltetramethylpentadienyl, menthyltetraphenylcyclopentadienyl, neomenthyl-tetraphenylcyclopentadienyl, menthylcyclopentadienyl, neomenthylcyclopentadienyl, tetrahydroindenyl, menthyltetrahydroindenyl and neomenthyltetrahydroindenyl groups. Pentamethylcyclopentadienyl is especially preferred.

When either A or B is an amide group represented by —NR⁴—, —NHR⁴, NR⁴R⁵, —NR⁸—, —NHR⁸ or NR⁷R⁸ wherein R⁶ and R⁷ are as hereinbefore defined, and where R⁴ or R⁸ is an acyl group represented by —C(O)R⁸ or —C(O)R⁹, R⁶ and R⁹ independently are often linear or branched C₁₋₇alkyl, C₁₋₈cycloalkyl or aryl, for example phenyl. Examples of acyl groups which may be represented by R⁴ or R⁹ include benzoyl, acetyl and halogenoacetyl, especially trifluoroacetyl groups.

When either A or B is present as a sulphonamide group represented by —NR⁴—, —NHR⁴, NR⁴R⁵, —NR⁸—, —NHR⁸ or NR⁷R⁸ wherein R⁵ and R⁷ are as hereinbefore defined, and where R⁴ or R⁸ is a sulphonyl group represented by —S(O)₂R⁶ or —S(O)₂R⁹, R⁶ and R⁹ independently are often linear or branched C₁₋₈alkyl, C₁₋₈cycloalkyl or aryl, for example phenyl. Preferred sulphonyl groups include methanesulphonyl, trifluoromethanesulphonyl and especially p-toluenesulphonyl groups and naphthylsulphonyl groups.

When either of A or B is present as a group represented by —NR⁴—, —NHR⁴, NR⁴R⁵, —NR⁸—, —NHR⁸ or NR⁷R⁸ wherein R⁵ and R are as hereinbefore defined, and where R⁸ or R⁸ is a group represented by C(O)NR⁸R¹⁰, C(S)NR⁸R¹⁰, C(═NR¹⁰)SR¹¹, C(═NR¹⁰)OR¹¹, C(O)NR⁹R¹², C(S)NR⁹R¹², C(═NR¹²)SR¹³ or C(═NR¹²)OR¹³, R⁶ and R⁹ independently are often linear or branched C₁₋₈alkyl, such as methyl, ethyl, isopropyl, C₁₋₈cycloalkyl or aryl, for example phenyl, groups and R¹⁰⁻¹³ are often each independently hydrogen or linear or branched C₁₋₈alkyl, such as methyl, ethyl, isopropyl, C₁₋₈cycloalkyl or aryl, for example phenyl, groups.

When B is present as a group represented by —OR⁷, —SR⁷, —PR⁷— or —PR⁷R⁹, R⁷ and R⁹ independently are often linear or branched C₁₋₈-alkyl, such as methyl, ethyl, isopropyl, C₁₋₈cycloalkyl or aryl, for example phenyl.

It will be recognised that the precise nature of A and B will be determined by whether A and/or B are formally bonded to the metal or are coordinated to the metal via a lone pair of electrons.

The groups A and B are connected by a linking group E. The linking group E achieves a suitable conformation of A and B so as to allow both A and B to bond or coordinate to the metal, M. A and B are commonly linked through 2, 3 or 4 atoms. The atoms in E linking A and B may carry one or more substituents. The atoms in E, especially the atoms alpha to A or B, may be linked to A and B, in such a way as to form a heterocyclic ring, preferably a saturated ring, and particularly a 5, 6 or 7-membered ring. Such a ring may be fused to one or more other rings. Often the atoms linking A and B will be carbon atoms. Preferably, one or more of the carbon atoms linking A and B will carry substituents in addition to A or B. Substituent groups include those which may substitute R⁵⁻⁷ or R⁹⁻¹¹ as defined above. Advantageously, any such substituent groups are selected to be groups which do not coordinate with the metal, M. Preferred substituents include halogen, cyano, nitro, sulphonyl, hydrocarbyl, perhalogenated hydrbcarbyl and heterocyclyl groups as defined above. Most preferred substituents are C₁₋₆ alkyl groups, and phenyl groups. Most preferably, A and B are linked by two carbon atoms, and especially an optionally substituted ethyl moiety. When A and B are linked by two carbon atoms, the two carbon atoms linking A and B may comprise part of an aromatic or aliphatic cyclic group, particularly a 5, 6 or 7-membered ring. Such a ring may be fused to one or more other such rings. Particularly preferred are embodiments in which E represents a 2 carbon atom separation and one or both of the carbon atoms carries an optionally substituted aryl group as defined above or E represents a 2 carbon atom separation which comprises a cyclopentane or cyclohexane ring, optionally fused to a phenyl ring.

E preferably comprises part of a compound having at least one stereospecific centre. Where any or all of the 2, 3 or 4 atoms linking A and B are substituted so as to define at (east one stereospecific centre on one or more of these atoms, it is preferred that at least one of the stereospecific centres be located at the atom adjacent to either group A or B. When at least one such stereospecific centre is present, it is advantageously present in an enantiomerically purified state.

When B represents —O— or —OH, and the adjacent atom in E is carbon, it is preferred that B does not form part of a carboxylic group.

Compounds which may be represented by A-E-B, or from which A-E-B may be derived by deprotonation, are often aminoalcohols, including 4-aminoalkan-1-ols, 1-aminoalkan-4-ols, 3-aminoalkan-1-ols, 1-aminoalkan-3-ols, and especially 2-aminoalkan-1-ols, 1-aminoalkan-2-ols, 3-aminoalkan-2-ols and 2-aminoalkan-3-ols, and particularly 2-aminoethanols or 3-aminopropanols, or are diamines, including 1,4-diaminoalkanes, 1,3-diaminoalkanes, especially 1,2- or 2,3-diaminoalkanes and particularly ethylenediamines. Further aminoalcohols that may be represented by A-E-B are 2-aminocyclopentanols and 2-aminocyclohexanols, preferably fused to a phenyl ring. Further diamines that may be represented by A-E-B are 1,2-diaminocycloperitanes and 1,2-diaminocyclohexanes, preferably fused to a phenyl ring. The amino groups may advantageously be N-tosylated. When a diamine is represented by A-E-B, preferably at least one amino group is N-tosylated. The aminoalcohols or diamines are advantageously substituted, especially on the linking group, E, by at least one alkyl group, such as a C₁₋₄-alkyl, and particularly a methyl, group or at least one aryl group, particularly a phenyl group.

Specific examples of compounds which can be represented by A-E-B and the protonated equivalents from which they may be derived are:

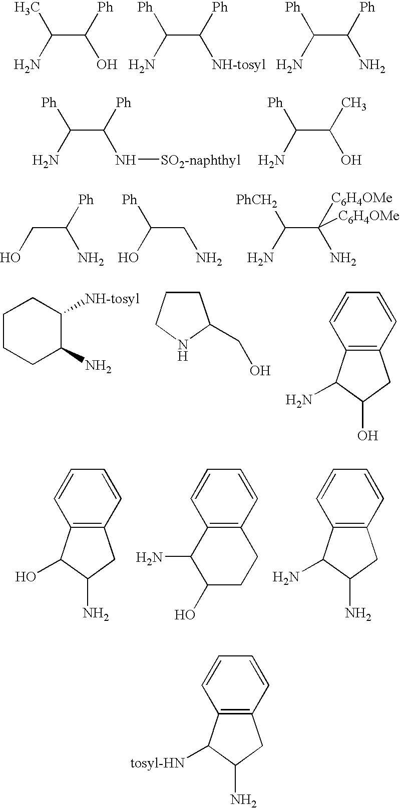

Preferably, the enantiomerically and/or diastereomerically purified forms of these are used. Examples include (1S,2R)-(+)-norephedrine, (1R,2S)-(+)-cis-1-amino-2-indanol, (1S,2R)-2-amino-1,2-diphenylethanol, (1S,2R)-(−)-cis-1-amino-2-indanol, (1R,2S)-(−)-norephedrine, (S)-(+)-2-amino-1-phenylethanol, (1R,2S)-2-amino-1,2-diphenylethanol, N-tosyl-(1R,2R)-1,2-diphenylethylenediamine, N-tosyl-(1S,2S)-1,2-diphenylethylenediamine, (1R,2S)-cis-1,2-indandiamine, (1S,2R)-cis-1,2-indandiamine, (R)-(−)-2-pyrrolidinemethanol and (S)-(+)-2-pyrrolidinemethanol.

Metals which may be represented by M include metals which are capable of catalysing transfer hydrogenation. Preferred metals include transition metals, more preferably the metals in Group VIII of the Periodic Table, especially ruthenium, rhodium or iridium. When the metal is ruthenium it is preferably present in valence state II. When the metal is rhodium or iridium it is preferably present in valence state I when R³ is a neutral optionally substituted hydrocarbyl or a neutral optionally substituted perhalogenated hydrocarbyl ligand, and preferably present in valence state III when R³ is an optionally substituted cyclopentadienyl ligand.

It is preferred that M, the metal, is rhodium present in valence state III and R³ is an optionally substituted cyclopentadienyl ligand.

Anionic groups which may be represented by Y include hydride, hydroxy, hydrocarbyloxy, hydrocarbylamino and halogen groups. Preferably when a halogen is represented by Y, the halogen is chloride. When a hydrocarbyloxy or hydrocarbylamino group is represented by Y, the group may be derived from the deprotonation of the hydrogen donor utilised in the reaction.

Basic ligands which may be represented by Y include water, C₁₋₄ alcohols, C₁₋₈ primary or secondary amines, or the hydrogen donor which is present in the reaction system. A preferred basic ligand represented by Y is water.

Most preferably, A-E-B, R³ and Y are chosen so that the catalyst is chiral. When such is the case, an enantiomerically and/or diastereomerically purified form is preferably employed. Such catalysts are most advantageously employed in asymmetric transfer hydrogenation processes. In many embodiments, the chirality of the catalyst is derived from the nature of A-E-B.

An especially preferred catalyst of Formula (A) is of formula:

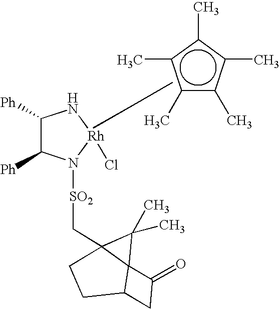

The preferred catalyst may be prepared in-situ preferably by combining a chiral bidentate nitrogen ligand with a Rh(III) metal complex containing a substituted cyclopentadienyl ligand. Preferably a solvent is present in this operation. The solvent used may be anyone which does not adversely effect the formation of the catalyst. These solvents include acetonitrile, ethylacetate, toluene, methanol, tetrahydrofuran, ethylmethyl ketone. Preferably the solvent is methanol.

Any suitable reductant may be used in the preferred embodiment of step (a), examples of reductants able to be used in this process include hydrogen donors including hydrogen, primary and secondary alcohols, primary and secondary amines, carboxylic acids and their esters and amine salts, readily dehydrogenatable hydrocarbons, clean reducing agents, and any combination thereof.

Primary and secondary alcohols which may be employed in the preferred embodiment of step (a) as hydrogen donors comprise commonly from 1 to 10 carbon atoms, preferably from 2 to 7 carbon atoms, and more preferably 3 or 4 carbon atoms. Examples of primary and secondary alcohols which may be represented as hydrogen donors include methanol, ethanol, propan-1-ol, propan-2-ol, butan-1-ol, butan-2-ol, cyclopentanol, cyclohexanol, benzylalcohol, and menthol, especially propan-2-ol and butan-2-ol.

Primary and secondary amines which may be employed in the preferred embodiment of step (a) as hydrogen donors comprise commonly from 1 to 20 carbon atoms, preferably from 2 to 14 carbon atoms, and more preferably 3 or 8 carbon atoms. Examples of primary and secondary amines which may act as hydrogen donors include ethylamine, propylamine, isopropylamine, butylamine, isobutylamine, hexylamine, diethylamine, dipropylamine, di-isopropylamine, dibutylamine, di-isobutylamine, dihexylamine, benzylamine, dibenzylamine and piperidine. When the hydrogen donor is an amine, primary amines are preferred, especially primary amines comprising a secondary alkyl group, particularly isopropylamine and isobutylamine.

Carboxylic acids and their esters which in a preferred embodiment of step (a) may act as hydrogen donors comprise commonly from 1 to 10 carbon atoms, preferably from 1 to 3 carbon atoms. In certain embodiments, the carboxylic acid is advantageously a beta-hydroxy-carboxylic acid. Esters may be derived from the carboxylic acid and a C₁₋₁₀ alcohol. Examples of carboxylic acids which may be employed as hydrogen donors include formic acid, lactic acid, ascorbic acid and mandelic acid, especially formic acid.

In certain preferred embodiments, when a carboxylic acid is employed as hydrogen donor, at least some of the carboxylic acid is preferably present as salt, preferably an amine, ammonium or metal salt. Preferably, when a metal salt is present the metal is selected from the alkali or alkaline earth metals of the periodic table, and more preferably is selected from the group I elements, such as lithium, sodium or potassium. Amines which may be used to form such salts include; primary, secondary and tertiary amines which comprise from 1 to 20 carbon atoms. Cyclic amines, both aromatic and non-aromatic, may also be used. Tertiary amines, especially trialkylamines, are preferred. Examples of amines which may be used to form salts include; trimethylamine, triethylamine, di-isopropylethylamine and pyridine. The most preferred amine is triethylamine.

When at least some of the carboxylic acid is present as an amine salt, particularly when a mixture of formic acid and triethylamine is employed; the mole ratio of acid to amine is between 1:1 and 50:1 and preferably between 1:1 and 10:1, and most preferably about 5:2. When at least some of the carboxylic acid is present as a metal salt, particularly when a mixture of formic acid and a group I metal salt is employed, the mole ratio of acid to metal ions present is between 1:1 and 50:1 and preferably between 1:1 and 10:1, and most preferably about 2:1. The ratios of acid to salts may be maintained during the course of the reaction by the addition of either component, but usually by the addition of the carboxylic acid.

Readily dehydrogenatable hydrocarbons which may be employed in step (a) as hydrogen donors comprise hydrocarbons which have a propensity to aromatise or hydrocarbons which have a propensity to form highly conjugated systems. Examples of readily dehydrogenatable hydrocarbons which may be employed by as hydrogen donors include cyclohexadiene, cyclohexene, tetralin, dihydrofuran and terpenes.

Clean reducing agents able to act as hydrogen donors comprise reducing agents with a high reduction potential, particularly those having a reduction potential relative to the standard hydrogen electrode of greater than about −0.1 eV, often greater than about −0.5 eV, and preferably greater than about −1 eV. Examples of suitable clean reducing agents include hydrazine and hydroxylamine.

The most preferred hydrogen donors in the preferred embodiment of step (a) are propan-2-ol, butan-2-ol, triethylammonium formate and a mixture of triethylammonium formate and formic acid.

Step (a) is preferably a stereospecific reaction. The predominant product may be either the R or S enantiomer of a compound of Formula (3) or Formula (7). The enantiomeric product of step (a) is preferably formed in at least 60% enantiomeric excess (e.e.), more preferably in at least 80% e.e and especially in at least 90% e.e.

The preferred product of step (a) Is a compound of Formula (9): 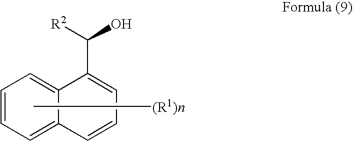
where R¹, R² and n are as defined for Formula (5).

Step (a) of the process may be performed in the presence of an organic solvent or mixture of organic solvents that is compatible with the reagents employed. These solvents include N,N-dimethylformamide, acetonitrile, tetrahydrofuran and C₁₋₄ alcohols such as methanol.

Step (a) of the process is performed at a temperature where the reactants and catalyst are sufficiently stable for the reaction to proceed to a significant degree. Preferably step (a) of the process is carried out at a temperature below 35° C. and more preferably in a range of from 0° C. to 20° C.

Step (a) of the process is advantageously allowed to proceed to at least 90% conversion, more preferably to at least 95% conversion.

The reaction time of step (a) of the process of the present invention will depend on a number of factors, for example the reagent concentrations, the relative amounts of reagents, the reaction temperature and particularly the presence and nature of any catalyst employed. Typical reaction times, in addition to the reagent addition times, range from 15 minute to 20 hours, with reaction times of 30 minutes to 10 hours being common.

Preferably, the process of step (a) is carried out under a substantially inert atmosphere, for example nitrogen or argon.

Compounds of Formula (2) and (6) may be purchased or prepared by methods well known in the art from commercially available starting materials. For example, 1-acetonaphthone may be purchased from Aldrich.

The leaving group donor in step (b) may be any compound known in the art able to react with the hydroxyl on compounds of Formula (3) and Formula (7) to give a species which may be displaced by ammonia and so yield a compound of Formula (1) and Formula (5).

The leaving group donor preferably forms an ester or a sulphonate bond with the hydroxyl group, especially a sulphonate bond.

Thus, the preferred leaving group donor is a compound of formula R¹⁴SO₂X, where R¹⁴ is an optionally substituted alkyl, optionally substituted aryl, such as phenyl or an optionally substituted heteroaryl group and X is a halogen.

It is especially preferred that R¹⁴ is optionally substituted C₁₋₄alkyl, particularly methyl.

X is preferably chloride.

Preferably the leaving group donor is methanesulphonyl chloride.

Step (b) of the process may be performed in the presence of an organic solvent or mixture of organic solvents which is unreactive towards the reagents employed. Examples of suitable solvents include toluene, tetrahydrofuran and acetonitrile.

Step (b) of the process is preferably performed at a temperature in the range of from −50° C. to 50° C. and more preferably in a range of from −20° C. to 20° C. It is especially preferred that step (b) is carried out at a temperature in the range of from −5° C. to 5° C.

Step (b) of the process is advantageously allowed to proceed to at least 90% conversion, more preferably to at least 95% conversion.

The reaction time of step (b) of the process of the present invention will depend on a number of factors, for example the reagent concentrations, the relative amounts of reagents and particularly the reaction temperature. Typical reaction times, in addition to the reagent addition times, range from 15 minute to 20 hours, with reaction times of 30 minutes to 10 hours being common.

Preferably, the process of step (b) is carried out under a substantially inert atmosphere, for example nitrogen or argon.

When the compounds of Formula (3) and Formula (7) are specific enantiomers step (b) is preferably carried out without any significant racemisation.

In step (c) of the process ammonia may be in any form able to react with compounds of Formula (4) and Formula (8) to give the corresponding amine. Preferably, ammonia is present as an aqueous solution.

Step (c) of the process may be performed in the presence of an organic solvent or mixture of organic solvents which is unreactive towards the reagents employed. Examples of suitable solvents include: tetrahydrofuran, toluene, acetonitrile, liquid ammonia and water.

Step (c) of the process is preferably performed at a temperature in the range of from −50° C. to 200° C. and more preferably in the range of from 0° C. to 180° C. It is especially preferred that step (b) is carried out in the range of from 40° C. to 140° C.

Step (c) of the process is preferably performed under a pressure in the range of from 1 to 100 bar and more preferably in the range of from 1 to 10 bar.

Step (c) of the process is advantageously allowed to proceed to at least 90% conversion, more preferably to at least 95% conversion.

The reaction time of step (c) of the process of the present invention will depend on a number of factors, for example the reagent concentrations, the relative amounts of reagents and particularly the pressure and reaction temperature. Typical reaction times, in addition to the reagent addition times, range from 15 minute to 20 hours, with reaction times of 30 minutes to 10 hours being common.

When the compound of Formula (4) or of Formula (8) is a specific enantiomer then step (c) is preferably carried out without any significant racemisation.

When the compound of Formula (1) or of Formula (5), formed by step (c), is a specific enantiomer then preferably it is formed in at least 60% enantiomeric excess (e.e.), more preferably in at least 80% e.e and especially in at least 90% e.e.

If the compound of Formula (1) or of Formula (5) is a sterioisomer it may be further purified, if necessary, by any method known in the art such as a diastereomeric salt resolution to enantioenrich the desired amine.

The compound of Formula (1) or of Formula (5) purified by diastereomeric salt resolution is preferably in at least 90% enantiomeric excess (e.e.), more preferably in at least 95% e.e and especially is in greater than 99% e.e.

A preferred embodiment of the first aspect of the invention provides a process for the preparation of a compound of Formula (10): 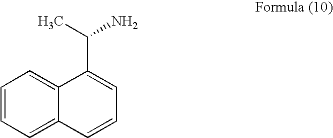
which comprises the steps:
(a) reducing a compound of Formula (11): 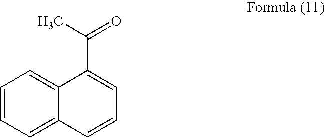
   to a compound of Formula (12): 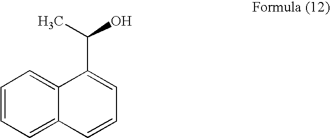
(b) reacting a compound of Formula (12) with a compound of formula R³SO₂X, in the presence of a base, to give a compound of Formula (13); 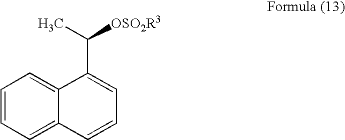
   wherein:

R³ is optionally substituted C₁₋₄alkyl; and

X is halogen:
(c) reacting a compound of Formula (13) with ammonia to give a compound of Formula (10).

Structural and process preferences for the preferred embodiment of the first aspect of the invention are as described above.

It is especially preferred that step (a) of this preferred embodiment is carried out in the presence of a catalyst of Formula (A) as described and preferred above.

When further purification of the compound of Formula (10) is required it may be achieved by any means known in the art. These methods include a diastereomeric salt resolution to enantioenrich the desired amine. Preferably the diastereomeric salt resolution employs (L)-tartaric acid or (L)-chloropropionic acid and more preferably (L)-chloropropionic acid.

The compound of Formula (10) purified by diastereomeric salt resolution is preferably in at least 90% enantiomeric excess (e.e.), more preferably in at least 95% e.e and especially is in greater than 99% e.e.

A second aspect of the invention proves a process for the preparation of a stereoisomer of a compound of Formula (14): 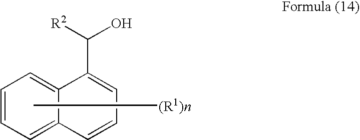
wherein R¹, R² and n are as described and preferred in the first aspect of the invention, which comprises the transfer hydrogenation of a compound of Formula (6): 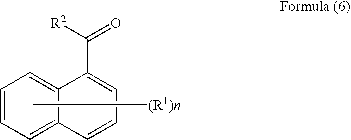
by a hydrogen donor in the presence of a catalyst of Formula (A): 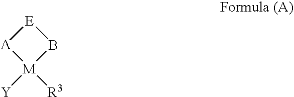
wherein the catalyst of Formula (A) and the hydrogen donor are as described and preferred in the first aspect of the invention.

A third aspect of the invention provides a process for the diastereomeric salt resolution of (S)-1-naphthylethylamine which comprises mixing (S)-1-naphthylethylamine with (2R,3R)-tartaric acid or (S)-chloropropionic acid, preferably (S)-chloropropionic acid, to form the corresponding diastereomeric salt. The diastereomeric salt so formed may be separated from the reaction mixture using established techniques such as filtration. Once isolated the diastereomeric salt may be further purified by repeating the process of the third aspect of the invention. The isolated diastereomeric salt may also be converted into other salt forms by established techniques known in the art such as ion-exchange chromatography and dialysis.

A fourth aspect of the invention provides a diastereomeric salt of (S)-1-naphthylethylamine with (2R,3R)-tartaric acid or (S)-chloropropionic acid, preferably (S)-chloropropionic acid.

A fifth aspect of the invention provides a compound of Formula (15): 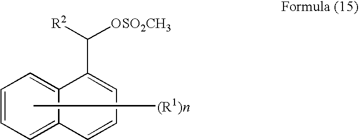
wherein R¹, R² and n are as preferred in the first aspect of the invention.

Preferably the compound of Formula (15) is of Formula (16):

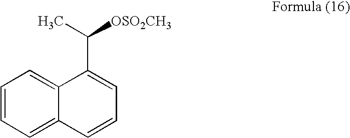

Compounds of Formula (15) may be formed by reacting a mesyl donor, such as methanesulfonyl chloride, with a compound of Formula (7) in the presence of an organic base, particularly triethylamine.

Many of the compounds described above may exist in the form of a salt. These salts are included within the scope of the present inventions.

The compounds described above may be converted to the salt form using known techniques.

The compounds described herein may exist in tautomeric forms other than those shown in this specification. These tautomers are also included within the scope of the present inventions.

The invention is illustrated, without limitation, by the following examples.

### EXAMPLE 1

Stage 1 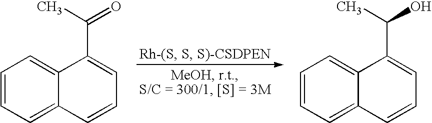
Stage 1
Selection of the Catalyst
Stage 1(a)
Selection of the Ligand

Ten mono-tosylated diamine ligands were screened to determine which would give the optimum stereo selective reduction of 1′-acetonaphthone to (R)-1-acetonaphthylethylalcohol.

In the experiments equivalents of [Rh pentamethylcyclopentadienylCl₂]₂ and the various mono-tosylated diamine ligands were added to tetrahydrofuran (THF) at room temperature with stirring under a low nitrogen purge for 30 mins. These catalyst solutions were added to 1′-acetonaphthone in a ratio of substrate to catalyst of 200 to 1. Formic acid (hydrogen donor) was then added slowly to the reaction mixtures, at a ratio of 6 to 1 formic acid to 1′-acetonaphthone. The reaction mixtures were left at room temperature for under nitrogen for 16 hours. At the end of this time the products were analysed by HPLC. The conditions were as follows:
Column: Chiralcel OD (25 cm×4.6 mm)
Eluent: Hexane/EtOH (absolute): 92.5/7.5
Flow rate: 1 mL/min
Detection: UV, 254 nm
Temperature: 30° C.

The ligands evaluated and the enantiomeric excess (e.e.) of the (R) enantiomer product are shown below in Table 1:

TABLE 1

| Ligand | Structure | e.e. (%) |
|---|---|---|
| A | 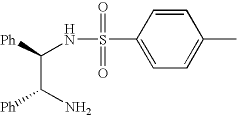 | 70 |
| B | 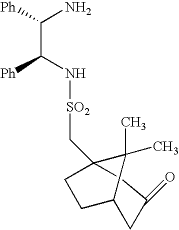 | 86 |
| C | 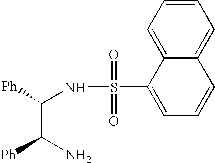 | 19 |
| D | 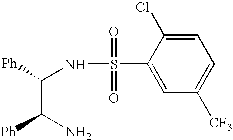 | 36 |
| E | 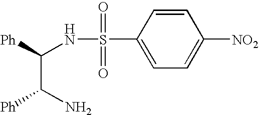 | 74 |
| F | 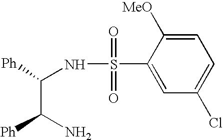 | 54 |
| G | 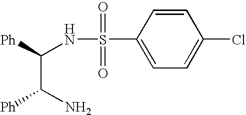 | 51 |
| H | 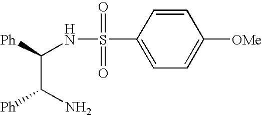 | 41 |
| I | 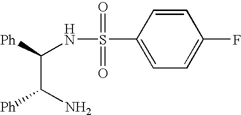 | 36 |
| J | 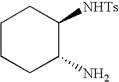 | 24 |

From the table above, it can be seen that the catalyst comprising ligand B ((S,S,S) CS-DPEN, ((S,S,S)-N-(2-Amino-1,2-diphenyl-ethyl)-C-(7,7-dimethyl-2-oxo-bicyclo[2.2.1]hept-1-yl)methanesulfonamide)) (CS-DPEN) is the most selective for (R)-1-acetonaphthylethylalcohol.

Stage 1(b)

Screening for the Optimum Solvent

The protocol of stage 1(a) was repeated using the CS-DPEN ligand (ligand B) in all cases but with tetrahydrofuran being replaced by the solvents as shown in Table 2. The optical purity of the products formed was determined using HPLC as described in stage 1 (a). Results are shown in Table 2 in terms of the enantiomeric excess (e.e.) of the (R) enantiomer of 1-acetonaphthylethylalcohol.

TABLE 2

| Solvent | e.e. (%) |
|---|---|
| Acrylonitrile | 76 |
| Ethylacetate | 84 |
| Toluene | 84 |
| Methanol | 95 |
| THF | 86 |
| Ethylmethyl ketone | 80 |

Table 2 shows that the solvent used in forming the catalyst has an effect on the stereo-selectivity of the reaction the best result being obtained with methanol.

Based on the results shown in Table 1 and Table 2 the (S,S,S)-CS-DPEN ligand was chosen as the catalyst ligand of choice and methanol was chosen as the preferred solvent for catalyst formation.
Stage 2 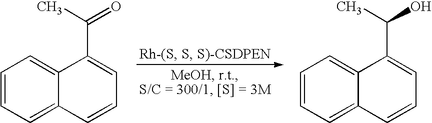
Stage 2 (a) Preparation of the Catalyst Solution

The catalyst was formed by adding [Rh pentamethylcyclopentadienylCl₂]₂ (60.5 mg), (S,S,S)-CS-DPEN (83.7 mg) and methanol (20 ml) to a round bottom-flask with stirring under a low nitrogen purge for 30 mins.

Stage 2(b)

1′-Acetonaphthone (10 g) was added to a 100 ml jacketed vessel and stirred for 15 minutes. The reactor temperature was set at 20° C. and the vessel was purged with nitrogen by continuous sparge and stirred throughout the reaction. One quarter of the catalyst solution prepared in stage 2(a) was added to the reaction vessel and then 13.8 ml of a mixture of triethylamine/formic acid (ratio 2:5) was added at a rate of 2.3 ml/min. One and a half hours after the first addition of the catalyst solution a further aliquot of one quarter of the catalyst solution was added to the reaction mixture and this was repeated after three and four and a half hours. The reaction mixture was allowed to stir at 20° C. for 12-18 hours until complete and then water (20 ml) was added in portions allowing the reaction temperature to warm to 20° C. in between additions. This mixture was transferred to a separating vessel at room temperature and toluene (40 ml) was added. The mixture was stirred vigorously for 30 minutes and then allowed to settle for 30 minutes. The organic layer was taken and brine (10%, 20 ml) was added. The mixture was again stirred vigorously for 30 minutes and then allowed to settle for a further 30 minutes. The extraction with brine was repeated two more times and then the organic solution was concentrated down to 20% volume by rotary evaporation. The reaction proceeded with greater than 99% conversion to give a product of 94.5% e.e.
Stage 3

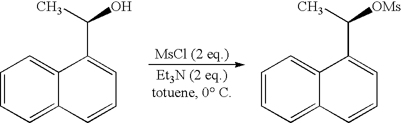

The product of stage 2 (10.1 g in 60 ml of toluene) was added to a reaction vessel and stirred under nitrogen. The reaction mixture was cooled to −5° C. and triethylamine (16.41 ml) was added dropwise. Methanesulfonyl chloride (9.28 ml) was then added dropwise while maintaining the temperature of the reaction mixture below 0° C. The reaction mixture was then allowed to warm to room temperature and stirred for a further 2.5 hours. The reaction mixture was then filtered to remove triethylamine hydrochloride and the resultant toluene solution was used directly in Stage 4.
Stage 4

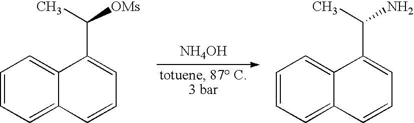

Aqueous ammonia (30%, 27.7 ml) was added to a Parr reactor. The toluene solution of the product of stage 3 was added and the reactor was sealed and heated to 87° C. at 3 bar and allowed to react for 5 hours. At the end of this time the pressure was released and the toluene solution was separated and then evaporated to dryness to yield the title product, (S)-1-naphthylethylamine, in 94% e.e. (S)-1-naphthylethylamine was assessed using the HPLC protocol as described in stage 1(a) where (S)-1-Naphthylethylamine eluted at 12.0 minutes and (R)-1-Naphthylethylamine eluted at 5.9 minutes.

Stage 5

Diastereomeric Salt Resolution

Stage 5a

Selection of the Salt Acid

Acids were screened to see which, in a diastereomeric salt resolution, would yield (S)-1-naphthylethylamine in the greatest optical purity. The following acids were evaluated; (L)-malic acid, (L)-mandelic acid, (L)-tartaric acid, (L)-chloropropionic acid (LCPA), (L)-camphor acid and (L)-camphorsulfonic acid. Each acid (except LCPA) was screened In a range of 4 solvents: ethanol/water, methanol/water, isopropyl/water, ethyl acetate.

(S)-1-Naphthylethylamine of 94% e.e., as produced in stage 4 was mixed with each of the above acids and the crystals which formed were collected and were analysed as described in stage 1(a) and stage 4.

| Acid | Solvent | Crystals | e.e (%) |
|---|---|---|---|
| (L)-malic acid | ethanol/water | Yes | 94 |
| | methanol/water | No | |
| | isopropyl alcohol/water | Yes | 94 |
| | ethylacetate | No | |
| (L)-Tartaric acid | ethanol/water | Yes | 97 |
| | methanol/water | Yes | 94 |
| | isopropyl alcohol/water | Yes | 95 |
| | ethylacetate | Yes | 94 |
| (L)-Chloropropionic acid | ethanol/water | Yes | >99 |
| (LCPA) | | | |
| (L)-Mandelic acid | ethanol/water | Yes | 95 |
| | methanol/water | Yes | 94 |
| | isopropyl alcohol/water | Yes | 94 |
| | ethylacetate | Yes | 94 |
| (L)-camphor acid | ethanol/water | Yes | 94 |
| | methanol/water | Yes | 94 |
| | isopropyl alcohol/water | Yes | 94 |
| | ethylacetate | No | |
| (L)-camphorsulfonic | ethanol/water | Yes | 94 |
| acid | methanol/water | No | |
| | isopropyl alcohol/water | No | |
| | ethylacetate | No | |

A significant improvement in e.e. was only observed with (L)-tartaric acid (97%) and with LCPA (>99%) in ethanol/water.

Stage 5(b)

Formation of the Salt

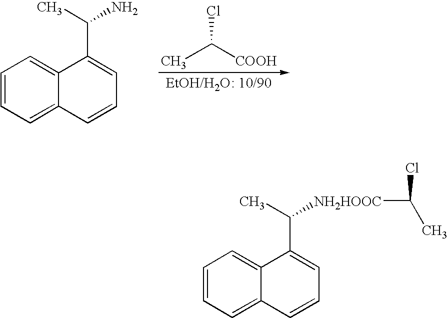

A mixture of ethanol (1.68 ml) and water (15.1 ml) was added with stirring to the product of stage 4. (L)-Chloropropionic acid (6.38 g, 1 equivalent of the product of step 4) was then added dropwise to the stirred mixture. The mixture was then heated to 60° C. and stirred for a further 30 minutes. The reaction mixture was cooled to room temperature and then concentrated to 50% by volume in a rotary evaporator and then allowed to settle until the precipitated salt is fully formed.

Step 5 (c)

Formation of the Free Amine

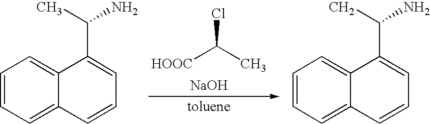

The salt from stage 5 (b) (4.53 g) was dissolved in 25 ml of 5M NaOH. Toluene (25 ml) was added to this solution while maintaining the pH above 10 by the addition of additional NaOH. The mixture was allowed to settle and the toluene solution was concentrated to dryness to yield the title product, as a yellow liquid, in greater than 99% e.e.

1. A process for the preparation of a compound of Formula (1): 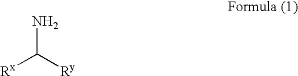
wherein:
R^{x} is optionally substituted aryl; and
R^{y} is optionally substituted hydrocarbyl:
which comprises the steps:
(a) reducing a compound of Formula (2): 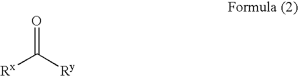
to a compound of Formula (3): 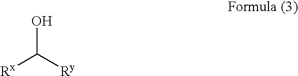
wherein R^{x} and R^{y} are as defined for Formula (1):
(b) reacting a compound of Formula (3) with a leaving group donor, to give a compound of Formula (4); 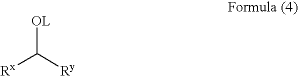
wherein:
R^{x} and R^{y} are as defined for Formula (1); and
OL is a leaving group:
(c) reacting a compound of Formula (4) with ammonia to give a compound of Formula (1).

2. A process according to claim 1 for the preparation of a compound of Formula (5): 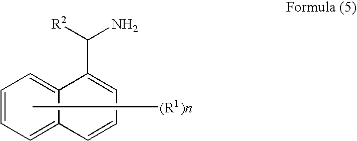
wherein:
R¹ is a substituent;
R² is optionally substituted hydrocarbyl; and
n is 0 to 4:
which comprises the steps:
(a) reducing a compound of Formula (6): 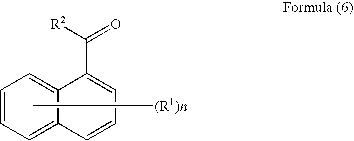
to a compound of Formula (7): 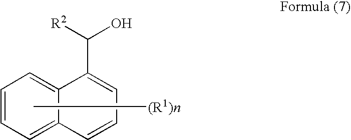
wherein R¹, R² and n are as defined for Formula (5):
(b) reacting a compound of Formula (7) with a leaving group donor, to give a compound of Formula (8); 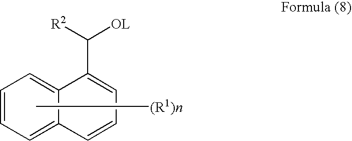
wherein:
R¹, R² and n are as defined for Formula (5);
OL is a leaving group:
(c) reacting a compound of Formula (8) with ammonia to give a compound of Formula (5).

3. A process according to claim 2 where R² is optionally substituted C₁₋₄alkyl.

4. A process according to claim 3 where R² is methyl.

5. A process according to claim 1 wherein n is 0.

6. A process according to claim 1 where step (a) is carried out in the presence of a catalyst.

7. A process according to claim 6 where the catalyst is of Formula (A): 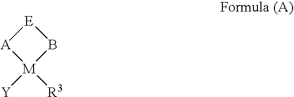
wherein:
R³ represents a neutral optionally substituted hydrocarbyl, a neutral optionally substituted perhalogenated hydrocarbyl, or an optionally substituted cyclopentadienyl ligand;
A represents —NR⁴—, —NR⁵—, —NHR⁴, —NR⁴R⁵ or —NR⁵R⁶ where R⁴ is H, C(O)R⁶, SO₂R⁶, C(O)NR⁶R¹⁰, C(S)NR⁶R¹⁰, C(═NR¹⁰)SR¹¹ or C(═NR¹⁰)OR¹¹, R⁵ and R⁶ each independently represents an optionally substituted hydrocarbyl, perhalogenated hydrocarbyl or an optionally substituted heterocyclyl group, and R¹⁰ and R¹¹ are each independently hydrogen or a group as defined for R⁶;
B represents —O—, —OH, OR⁷, —S—, —SH, SR⁷, —NR⁷—, —NR⁸—, —NHR⁸, —NR⁷R⁸, —NR⁷R⁹, —PR⁷— or —PR⁷R⁹ where R⁸ is H, C(O)R⁹, SO₂R⁹, C(O)NR⁹R¹², C(S)NR⁹R¹², C(═NR¹²)SR¹³ or C(═NR¹²)OR¹³, R⁷ and R⁹ each independently represents an optionally substituted hydrocarbyl, perhalogenated hydrocarbyl or an optionally substituted heterocyclyl group, and R¹² and R¹³ are each independently hydrogen or a group as defined for R⁹;
E represents a linking group;
M represents a metal capable of catalysing transfer hydrogenation; and
Y represents an anionic group, a basic ligand or a vacant site;
provided that when Y is not a vacant site that at least one of A or B carries a hydrogen atom.

8. A process according to claim 7 wherein A-E-B, R³ and Y are chosen so that the catalyst is chiral.

9. A process according to claim 7 wherein M, the metal, is rhodium present in valence state III and R³ is an optionally substituted cyclopentadienyl ligand.

10. A process according to claim 7 where the catalyst of Formula (A) is of formula:

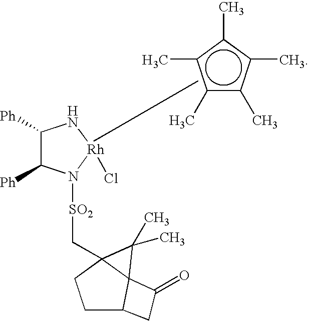

11. A process according to claim 1 wherein step (a) is a stereospecific reaction.

12. A process according to claim 1 wherein the product of step (a) is a compound of Formula (9): 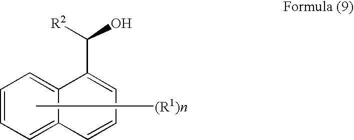
wherein:
R¹ is a substituent;
R² is optionally substituted hydrocarbyl; and
n is 0 to 4.

13. A process according to claim 1 where in step (b) the leaving group donor is a compound of formula R¹⁴SO₂X, where R¹⁴ is an optionally substituted alkyl, optionally substituted aryl or an optionally substituted heteroaryl group and X is a halogen.

14. A process according to claim 13 where in step (b) the leaving group donor is methanesulphonyl chloride.

15. A process according to claim 2 for the preparation of a compound of Formula (10): 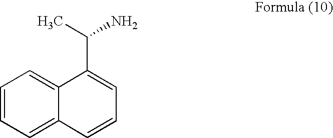
which comprises the steps:
(a) reducing a compound of Formula (11): 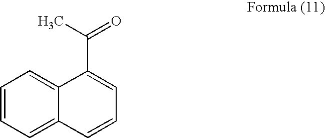
to a compound of Formula (12): 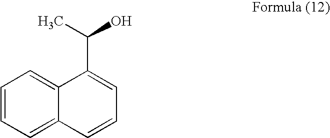
(b) reacting a compound of Formula (12) with a compound of formula R³SO₂X, in the presence of a base, to give a compound of Formula (13); 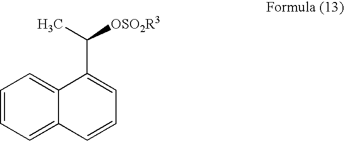
wherein:
R³ is optionally substituted C₁₋₄alkyl; and
X is halogen:
(c) reacting a compound of Formula (13) with ammonia to give a compound of Formula (10).

16. A process according to claim 15 where step (a) is carried out in the presence of a catalyst of Formula (A) as described in claim 7.

17. A process according to claim 15 wherein the compound of Formula (10) is purified by diastereomeric salt resolution using (L)-tartaric acid or (L)-chloropropionic acid.

18. A process for the preparation of a stereoisomer of a compound of Formula (14): 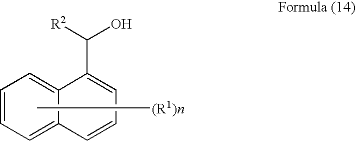
wherein:
R¹ is a substituent;
R² is optionally substituted hydrocarbyl; and
n is 0 to 4:
which comprises the transfer hydrogenation of a compound of Formula (6): 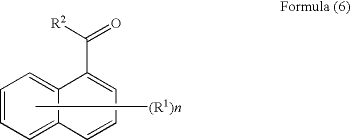
by a hydrogen donor in the presence of a catalyst of Formula (A) as described in claim 7.

19. A process for the diastereomeric salt resolution of (S)-1-naphthylethylamine which comprises mixing (S)-1-naphthylethylamine with (2R,3R)-tartaric acid or (S)-chloropropionic acid to form the corresponding diastereomeric salt.

20. A diastereomeric salt of (S)-1-naphthylethylamine with (2R,3R)-tartaric acid or (S)-chloropropionic acid.

21. A compound of Formula (15): 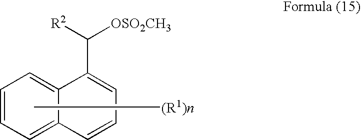
wherein:
R¹ is a substituent;
R² is optionally substituted hydrocarbyl; and
n is 0 to 4.

22. A compound according to claim 21 of Formula (15) which is of Formula (16): [chemical structure]
